(19) United States
(12) Patent Application Publication
(10) Pub. No.: US 2026/0146288 A1
(43) Pub. Date: May 28, 2026

(54) METHODS FOR DETERMINING GUT MICROBIOME HEALTH

(71) Applicant: Modgut Co., Ltd., Bangkok (TH)

(72) Inventors: Sawannee Sutheeworapong, Bangkok (TH); Pinidphon Prombutara, Bangkok (TH); Titawadee Kiatnikorn, Bangkok (TH); Nijaporn Chongudomliuk, Bangkok (TH)

(73) Assignee: Modgut Co., Ltd., Bangkok (TH)

(21) Appl. No.: 19/398,039

(22) Filed: Nov. 24, 2025

Related U.S. Application Data
(60) Provisional application No. 63/724,436, filed on Nov. 25, 2024.

(51) Int. Cl. C12Q 1/6883; G16B 30/00; G16B 40/00; G16H 10/40; G16H 20/00

(52) U.S. Cl. CPC C12Q 1/6883; G16B 30/00; G16B 40/00; G16H 10/40; G16H 20/00; C12Q 2600/156

(57) ABSTRACT

Methods comprising obtaining a biological sample and extracting nucleic acids from it are disclosed. The methods process the nucleic acids for DNA sequencing and identify in the DNA sequencing data a plurality of amplicon sequence variants (ASV). The methods record the relative abundance of each ASV and classify, using a machine learning algorithm, each ASV according to one of a set of taxonomies. The set of taxonomies includes a pathobiont bacteria subset consisting of genus level groups Eggerthella group, Ruminococcus torques group, Ruminococcus gnavus group, Coprobacillus group, Streptococcus group, Bilophila group, Actinomyces group, Desulfovibrio group, Atopobiaceae group, Veillonella group, Enterococcus group, Escherichia-Shigella group, Campylobacter group, Enterobacteriaceae group, and Fusobacterium group. Based on the relative abundance of the ASV classified as pathobiont bacteria, a numerical indication of the health of the subject is calculated. Based on the numerical indication, the methods deliver to the subject a customized intervention.

### CROSS REFERENCE TO RELATED APPLICATION

This application claims priority to U.S. Provisional Application No. 63/724,436 entitled “Methods for Determining Gut Microbiome Health” with a filing date of Nov. 25, 2024.

### TECHNICAL FIELD

This disclosure is related to the field of medicine, and more specifically medical diagnostics and methods of determining optimal treatment for disease or abnormality, including gut dysbiosis, non-alcoholic fatty liver disease and leaky gut syndrome. In particular, this disclosure describes systems and methods for determining gut microbiome health and calculating a numerical indication of the health of a subject.

### BACKGROUND

Trillions of microorganisms live in the human gastrointestinal tract, collectively known as the gut microbiota. These microorganisms include bacteria, viruses, fungi, and other microbes. They play a crucial role in various aspects of human health, including indigestible food degradation, immune function, mood regulation, and pathogen growth prevention. They help dietary fiber digestion and then produce short-chain fatty acids mainly including butyrate, propionate, and acetate. They also produce neurotransmitters, i.e., serotonin, gaba, and melatonin, by digesting amino acids through the tryptophan pathway (Gao et al., 2020).

However, a high-fat diet, low fiber intake, and consumption of processed foods can contribute to an imbalance condition, known as gut dysbiosis, by promoting the growth of pathobionts and pathogenic bacteria while reducing the populations of beneficial microorganisms. Pathobionts gradually injure the human body through metabolite production. Prolonged presence of harmful metabolites in the human body can result in chronic unhealthy conditions. Gut dysbiosis can disrupt the normal functioning of the gut and has been associated with unfavorable conditions such as obesity, inflammatory bowel disease, and even mental health disorders. Therefore, to determine when it is necessary to terminate a gut dysbiosis state before developing chronic diseases, disclosed herein is an indicator identifying the level of imbalance between beneficial bacteria and harmful, or pathobiont, bacteria, also called a gut dysbiosis score. The indicator has been tested with gut microbiota profiles of Thai individuals and validated by comparison with age, obesity, and disease-related variables.

The following documents are incorporated herein by reference: Barton L L, Ritz N L, Fauque G D, Lin H C. Sulfur cycling and the intestinal microbiome. Dig Dis Sci. 2017; 62:2241-2257. Biagi E, Franceschi C, Rampelli S, Severgnini M, Ostan R, Turroni S, et al. Gut microbiota and extreme longevity. Curr Biol. 2016; 26(11):1480-5. https://doi.org/10.1016/j.cub.2016.04.016. Franceschi C, Bonafë M, Valensin S, Olivieri F, De Luca M, Ottaviani E, et al. Inflamm-aging: an evolutionary perspective on immunosenescence. Ann N Y Acad Sci. 2000; 908(1):244-54. https://doi.org/10.1111/j.1749-6632.2000.tb06651.x. Gao K, Mu C L, Farzi A, Zhu W Y. Tryptophan metabolism: A link between the gut microbiota and brain. Adv Nutr. 2020 May 1; 11(3):709-723. https://doi.org/10.1093/advances/nmz127. PMID: 31825083; PMCID: PMC7231603. Ghosh T S, Das M, Jeffery I B, O'Toole P W. Adjusting for age improves identification of gut microbiome alterations in multiple diseases. eLife. 2020; 9. https://doi.org/10.7554/eLife.50240. Glover J S, Ticer T D, Engevik M A. Characterizing the mucin-degrading capacity of the human gut microbiota. Sci Rep. 2022; 12:8456. https://doi.org/10.1038/s41598-022-11819-z. Hale V L, Jeraldo P, Mundy M, Yao J, Keeney G, Scott N, Cheek E H, Davidson J, Greene M, Martinez C, et al. Synthesis of multi-omic data and community metabolic models reveals insights into the role of hydrogen sulfide in colon cancer. Methods. 2018; 149:59-68. Ijssennagger N, Belzer C, Hooiveld G J, Dekker J, van Mil S W, Müller M, Kleerebezem M, van der Meer R. Gut microbiota facilitates dietary heme-induced epithelial hyperproliferation by opening the mucus barrier in colon. Proc Natl Acad Sci USA. 2015; 112:10038-10043. Kim K A, Jeong J J, Yoo S Y, Kim D H. Gut microbiota lipopolysaccharide accelerates inflamm-aging in mice. BMC Microbiol. 2016; 16(1):1-9. https://doi.org/10.1186/s12866-016-0625-7. Molinero N, Antón-Fernández A, Hernández F, Avila J, Bartolome B, Moreno-Arribas M V. Gut microbiota, an additional hallmark of human aging and neurodegeneration. Neuroscience. 2023 May 10; 518:141-161. https://doi.org/10.1016/j.neuroscience.2023.02.014. Epub 2023 Mar. 8. PMID: 36893982. Sovran B, Hugenholtz F, Elderman M, Van Beek A A, Graversen K, Huijskes M, et al. Age-associated impairment of the mucus barrier function is associated with profound changes in microbiota and immunity. Sci Rep. 2019; 9(1):1437. https://doi.org/10.1038/s41598-018-35228-3. Tomasova L, Konopelski P, Ufnal M. Gut bacteria and hydrogen sulfide: The new old players in circulatory system homeostasis. Molecules. 2016; 21:1558.

### SUMMARY

Disclosed herein are methods comprising obtaining at least one biological sample from at least one subject and extracting nucleic acids from the biological sample. The methods also process the nucleic acids for DNA sequencing, thereby generating DNA sequencing data and identify, by a computer system, in the DNA sequencing data in electronic form a plurality of amplicon sequence variants. The methods record the relative abundance of each amplicon sequence variant in a corresponding row of a relative abundance table and classify, using a machine learning algorithm, each amplicon sequence variant according to an associated taxonomy selected from a set of taxonomies. The set of taxonomies includes a pathobiont bacteria subset consisting of genus level groups Eggerthella group, Ruminococcus torques group, Ruminococcus gnavus group, Coprobacillus group, Streptococcus group, Bilophila group, Actinomyces group, Desulfovibrio group, Atopobiaceae group, Veillonella group, Enterococcus group, Escherichia-Shigella group, Campylobacter group, Enterobacteriaceae group, and Fusobacterium group. The methods calculate, by the computer system, and based on the relative abundance of the amplicon sequence variants classified as pathobiont bacteria in the biological sample, a numerical indication of the health of the subject. Based on the numerical indication, the methods deliver to the subject a customized intervention to improve the health of the subject. The intervention includes at least one of a dietary recommendation, a probiotic supplement recommendation, a lifestyle adjustment recommendation or a medical intervention recommendation.

In some embodiments the subject is a human and/or the biological sample is a stool sample. The customized intervention may include a written report describing the intervention and including information about the subject, the numerical indication, information about gut microbiome homeostasis, information about gut microbiome imbalance, and information about gut microbiome diversity. In some embodiments the method may include setting a maximum threshold of the sum of the relative abundance of the amplicon sequence variants associated with the pathobiont subset. Calculating the numerical indication may include assessing the range of the numerical indication to be from zero to the numerical indication based on the maximum threshold; and further include associating, with a first dietary recommendation, the probiotic supplement recommendation, and the lifestyle adjustment recommendation, a first portion of the range of the numerical indication starting from zero. The methods may also associate, with a second dietary recommendation, the probiotic supplement recommendation, and the lifestyle adjustment recommendation a second portion of the range of the numerical indication starting from the first portion. A third dietary recommendation, the probiotic supplement recommendation, and the lifestyle adjustment recommendation may be associated with a third portion of the range of the numerical indication starting from the second portion. And the methods may also associate, with a fourth dietary recommendation, the probiotic supplement recommendation, the lifestyle adjustment recommendation, and the medical intervention recommendation, a fourth portion of the range of the numerical indication starting from the third portion. A fifth dietary recommendation, the probiotic supplement recommendation, the lifestyle adjustment recommendation, and the medical intervention recommendation, may be associated with a fifth portion of the range of the numerical indication starting from the fourth portion. In some embodiments the methods include normalizing the relative abundances of the amplicon sequence variants associated with the pathobiont subset.

Also disclosed herein are methods of diagnosing non-alcoholic fatty liver disease comprising receiving at least one biological sample from at least one subject and extracting nucleic acids from the biological sample. The methods also process the nucleic acids for DNA sequencing, thereby generating DNA sequencing data and identify, by a computer system, in the DNA sequencing data in electronic form a plurality of amplicon sequence variants. The methods record the relative abundance of each amplicon sequence variant in a corresponding row of a relative abundance table and classify, using a machine learning algorithm, each amplicon sequence variant according to an associated taxonomy selected from a set of taxonomies. The set of taxonomies includes a pathobiont bacteria subset. The pathobiont bacteria subset consists of genus level groups Veillonella group, Alistipes group, Megamonas group, Dorea group, Robinsoniella group, Parabacteroides group, Allisonella group, and Escherichia-Shigella group. The methods calculate, by the computer system, based on the relative abundance of the amplicon sequence variants classified as pathobiont bacteria in the biological sample, a numerical indication of health of the subject. The numerical indication may fall into a range indicating the subject has non-alcoholic fatty liver disease or a second range indicating that the subject does not have non-alcoholic fatty liver disease. Based on the numerical indication, the methods deliver to the subject a customized intervention to improve the health of the subject. The intervention includes at least one of a dietary recommendation, a probiotic supplement recommendation, a lifestyle adjustment recommendation or a medical intervention recommendation. The medical intervention recommendation includes a recommendation to treat non-alcoholic fatty liver disease.

In some embodiments the subject is a human and the biological sample is a stool sample. In some embodiments the dietary recommendation is based on a Thai diet and/or includes functional foods found in an Asian diet. The customized intervention may include a written report describing the intervention and including information about the subject, the numerical indication, information about gut microbiome homeostasis, information about gut microbiome imbalance, information about gut microbiome diversity, and information about non-alcoholic fatty liver disease. Calculating the numerical indication may include assessing the normal range of the relative abundance based on a reference population; and if the numerical indication is below the normal range then providing a first dietary recommendation, the probiotic supplement recommendation, and the lifestyle adjustment recommendation; or if the numerical indication is in the normal range then providing a second dietary recommendation, the probiotic supplement recommendation, and the lifestyle adjustment recommendation; and if the numerical indication is above the normal range then providing a third dietary recommendation, the probiotic supplement recommendation, the lifestyle adjustment recommendation, and the medical intervention recommendation.

Also disclosed herein are methods of diagnosing leaky gut syndrome comprising obtaining at least one biological sample from at least one subject and extracting nucleic acids from the biological sample. The methods also process the nucleic acids for DNA sequencing, thereby generating DNA sequencing data and identify, by a computer system, in the DNA sequencing data in electronic form a plurality of amplicon sequence variants. The methods record the relative abundance of each amplicon sequence variant in a corresponding row of a relative abundance table and classify, using a machine learning algorithm, each amplicon sequence variant according to an associated taxonomy selected from a set of taxonomies. The set of taxonomies includes a pathobiont bacteria subset. The pathobiont bacteria subset consists of genus level groups Eggerthella group, Ruminococcus torques group, Ruminococcus gnavus group, Coprobacillus group, Streptococcus group, Bilophila group, Actinomyces group, Desulfovibrio group, Atopobiaceae group, Veillonella group, Enterococcus group, Escherichia-Shigella group, Campylobacter group, Enterobacteriaceae group, and Fusobacterium group. The methods calculate, by the computer system, based on the relative abundance of amplicon sequence variants classified as pathobiont bacteria in the biological sample, a numerical indication of health of the subject. The numerical indication may fall into a subset indicating the subject has leaky gut syndrome or a second subset indicating that the subject does not have leaky gut syndrome. Based on the numerical indication, the methods deliver to the subject a customized intervention to improve the health of the subject. The intervention includes at least one of a dietary recommendation, a probiotic supplement recommendation, a lifestyle adjustment recommendation or medical intervention recommendation. The medical intervention recommendation includes a recommendation to treat leaky gut syndrome.

In some embodiments, the subject is a human and the biological sample is a stool sample. The customized intervention may include a written report describing the intervention and including information about the subject, the numerical indication, information about gut microbiome homeostasis, information about gut microbiome imbalance, information about gut microbiome diversity, and information about leaky gut syndrome. Some embodiments include setting a maximum of the sum of the relative abundance of the amplicon sequence variants associated with the pathobiont subset and calculating the numerical indication may include assessing the range of the numerical indication from zero to the numerical indication based on the maximum of the sum of the relative abundance. The calculation may also include associating, with a first dietary recommendation, the probiotic supplement recommendation, and the lifestyle adjustment recommendation, a first portion of the range of the numerical indication starting from zero. The methods may also associate, with a second dietary recommendation, the probiotic supplement recommendation, and the lifestyle adjustment recommendation, a second portion of the range of the numerical indication starting from the first portion. A third dietary recommendation, the probiotic supplement recommendation, and the lifestyle adjustment recommendation may be associated with a third portion of the range of the numerical indication starting from the second portion. A fourth dietary recommendation, the probiotic supplement recommendation, the lifestyle adjustment recommendation, and the medical intervention recommendation may be associated with a fourth portion of the range of the numerical indication starting from the third portion. And the methods may associate, with a fifth dietary recommendation, the probiotic supplement recommendation, the lifestyle adjustment recommendation, and the medical intervention recommendation, a fifth portion of the range of the numerical indication starting from the fourth portion. In some embodiments, the methods include normalizing the sum of the relative abundance of the amplicon sequence variants associated with the pathobiont subset. The methods may also include evaluating the relative abundances of Bacteroides thetaiotaomicron, Enterobacteriaceae, Desulfovibrio, Bilophila, Ruminococcus gnavus, Clostridium sensu stricto 1, Lachnoclostridium, Eggerthella, Anaerotruncus, and Peptococcaceae.

### BRIEF DESCRIPTION OF THE DRAWINGS

Now referring to the attached drawings which form a part of this original disclosure:

FIG. 1 is a flow chart showing a method according to an embodiment.

FIG. 2 is a flow chart showing a method of diagnosing non-alcoholic fatty liver disease according to an embodiment.

FIG. 3 is a flow chart showing a method of diagnosing leaky gut syndrome according to an embodiment.

FIG. 4 is a graph showing the relationship between a numerical indication of the health of the subject and age group with p value.

FIG. 5 is a graph showing the relationship between a numerical indication of the health of the subject and BMI group with p value.

### DETAILED DESCRIPTION

Disclosed herein are methods, in particular methods for determining gut microbiome health, diagnosing non-alchoholic fatty liver disease, and leaky gut syndrome.

Unless otherwise defined below, all technical and scientific terms used herein have the same meaning as commonly understood by one of ordinary skill in the art. If there is an apparent discrepancy between the usage of a term in the art and its definition provided herein, the definition provided within the specification shall prevail.

“Microbiota” refers to the community of microorganisms that occur (sustainably or transiently) in and on an animal subject, typically a mammal such as a human, including eukaryotes, archaea, bacteria, and viruses (including bacterial viruses i.e., phages).

“Microbiome” refers to the genetic content of the communities of microbes that live in and on the human body, both sustainably and transiently, including eukaryotes, archaea, bacteria, and viruses (including bacterial viruses (i.e., phages)), wherein “genetic content” includes genomic DNA, RNA such as ribosomal RNA, the epigenome, plasmids, and all other types of genetic information.

As used herein, the term “gut” is understood to refer to the human gastrointestinal tract, also known as the alimentary canal. The gut includes the mouth, pharynx, oesophagus, stomach, small intestine (duodenum, jejunum, ileum), large intestines (cecum and colon) and rectum. While the entire alimentary canal can be colonized by varying species of microbes, the majority of the gut microbiome, in terms of both numbers of species of biomass, resides in the intestines (small and large).

As used herein, “bacteria” or “bacterial strain” is understood to mean a species or related taxonomic group of bacteria. A “bacterium” is understood as a single bacterial cell of a given species or related taxonomic group of bacteria.

“Dysbiosis” refers to a state of the microbiota of the gut, or other body area in a subject, including mucosal or skin surfaces, in which the normal diversity and/or function of the ecological network is disrupted. This unhealthy state can be due to a decrease in diversity, the overgrowth of one or more pathogens or pathobionts, symbiotic organisms able to cause disease only when certain genetic and/or environmental conditions are present in a subject, or the shift to an ecological microbial network that no longer provides an essential function to the host subject, and therefore no longer promotes health.

“Pathobionts” or “Opportunistic Pathogens” refers to symbiotic organisms able to cause disease only when certain genetic and/or environmental conditions are present in a subject.

The term “subject” refers to any animal subject including humans, laboratory animals (e.g., primates, rats, mice), livestock (e.g., cows, sheep, goats, pigs, turkeys, and chickens), and household pets (e.g., dogs, cats, and rodents). The subject may be suffering from a dysbiosis, including, but not limited to, an infection due to a gastrointestinal pathogen or may be at risk of developing or transmitting to others an infection due to a gastrointestinal pathogen.

“Stool sample” and “fecal sample” are used interchangeably and refer to a sample or aliquot of the stool or feces of a subject.

“Prebiotics” are non-digestible food ingredients that stimulate the growth and/or activity of bacteria in the digestive system in ways claimed to be beneficial to health. They often are selectively fermented ingredients that allow specific changes, both in the composition and/or activity of the gut microbiota.

“Probiotics” are micro-organisms that have claimed health benefits when consumed. Probiotics are commonly consumed as part of fermented foods with specially added active live cultures, such as in yogurt, soy yogurt, and the like, or as dietary supplements. Generally, probiotics help gut microbiota keep (or re-find) its balance, integrity and diversity. The effects of probiotics are usually strain-dependent.

“Synbiotics” refer to nutritional supplements combining probiotics and prebiotics in a form of synergism, hence synbiotics.

An “Asian diet” as used herein, refers to a diet rich in foods influenced by the cuisines of Asian countries such as China, India, Japan, Korea and Thailand. An Asian diet is typically rich in herbs and spices and centers dishes eaten with or comprising rice. An Asian diet may include Peking Duck (also known as Beijing Roasted Duck), Kung Pao Chicken, Sweet and Sour Pork, Hot Pot, Cantonese Dim Sum, Dumplings, Ma Po Tofu, Cantonese Char Siu, Biriyani, Pani Puri, Samosas, Falooda, Indian Curries, Sushi, Sashimi, Miso, Ramen, Soba, Shabu-shabu, Okonomiyaki, Tempura, Kaiseki, Natto, Kimchi, Kimchi fried rice, Sundubu jjigae, bulgogi, Tteokbokki, Japchae, Kan poong gi, Pad Thai, Som Tum, Tom Yum Soup, Tom Kha Gai, Khao Pad (also known as Thai fried rice), Pad Kra Pow Gai (also known as chicken with holy basil), Green Curry Chicken, Chicken with Cashew Nuts, Thai Red Curry, Pad See Ew, Ka Pi, Boodu, and Tempeh. Other foods and dishes may also be included in an Asian diet as known to one skilled in the art.

A “Thai diet” as used herein, includes Northern, Northeastern, Central and Southern Thai cuisine. A Thai diet often uses coconut, garlic and onion and is rich in Thai herbs, including holy basil, lemon basil, sweet basil, bay leaves, coriander root, mint, parsley, pandan, and Thai celery. Northern Thai cuisine includes Khao Soi, Khanom Jeen Nam Ngiao, Hunglay Curry, Nam Prik Noom, Sai Oa sausage, Khao Tom Hua Ngok, and Khanom Pard, and other dishes known to those skilled in the art. Northeastern Thai cuisine includes Som Tum, Isaan Fermented Sausage, Bamboo Shoot Curry, Minced Meat Salad (also known as Laab), Gai Yang, Duck Laab, Weeping Tiger Steak, Gaeng Om Gai, Soop Naw Mai, and Jaew sauce, and other dishes known to those skilled in the art. Central Thai cuisine includes Pad Thai, Som Tum, Tom Yum Soup, Tom Kha Gai, Khao Pad (also known as Thai fried rice), Pad Kra Pow Gai (also known as chicken with holy basil), Green Curry Chicken, Chicken with Cashew Nuts, Thai Red Curry, and Pad See Ew, and other dishes known to those skilled in the art. Southern Thai cuisine includes Gaeng Som Pla (also known as orange curry with fish), Gaeng Tai Pla, Kua Kling, Tom Som Pla Krabok, Gaeng Sataw, Khao Yam, and Nam Prik Goong Siap, and other dishes known to those skilled in the art. Other foods and dishes may also be included in a Thai diet as known to one skilled in the art.

Methods described herein are implemented using a “computer system” comprising computer programs executing on programmable computers, each comprising at least one processor, a data storage system (including volatile and non-volatile memory and/or storage elements), at least one input device, and at least one output device. For example, and without limitation, the programmable computer may be a programmable logic unit, a mainframe computer, server, personal computer, cloud-based program or system, laptop, personal data assistance, cellular telephone, smartphone, or tablet device.

Each program is preferably implemented in a high-level procedural or object-oriented programming and/or scripting language to communicate with the computer system. However, the programs can be implemented in assembly or machine language, if desired. In any case, the language may be a compiled or interpreted language. Each such computer program is preferably stored on a storage media or a device readable by a general or special purpose programmable computer for configuring and operating the computer when the storage media or device is read by the computer to perform the procedures described herein. The methods disclosed herein may be implemented partially or wholly via programs.

The term “consisting of” refers to compositions, methods, and respective components thereof as described herein, which are exclusive of any element not recited in that description of the embodiment.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like elements. While examples and features of disclosed principles are described herein, modifications, adaptations, and other implementations are possible without departing from the scope of the disclosed embodiments.

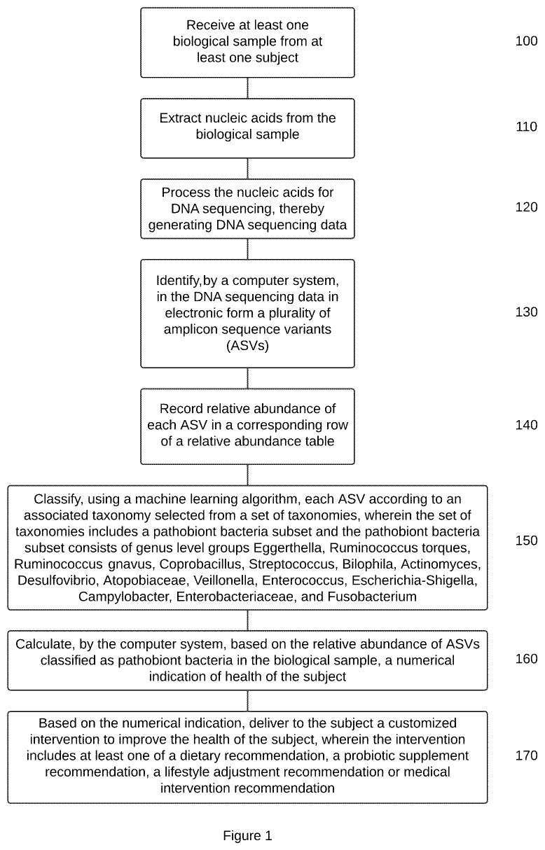

The methods disclosed herein include receiving at least one biological sample from at least one subject 100, as shown in FIGS. 1-3. In various embodiments, the methods use a biological sample that includes microbes obtained from the subject. In some embodiments, the biological sample is a gut, stool, or fecal sample obtained by non-invasive or invasive techniques such as biopsy of a subject. In one aspect, a stool or fecal sample refers to any preparation derived from fecal matter of a subject. Biological samples also include, but are not limited to, body fluid (e.g., blood, blood plasma, serum, or urine), secretions (e.g. sweat, vaginal secretions), organs, tissues, fractions, and cells isolated from mammals including, humans. For example, a sample of biological material obtained using a non-invasive method can be used to isolate nucleic acid molecules for the methods disclosed herein. In some embodiments, biological secretions are obtained from the digestive tract. Solid samples may be liquefied or mixed with a solution, and then genetic material may be extracted in accordance with any nucleic acid extraction protocols known in the art. In some embodiments, the extracted genetic material may be subjected to further processing and analysis, such as purification, amplification, and sequencing. In some embodiments, the extracted genetic material is subjected to metagenomics analysis to, for example, identify the one or more types of microbiota in the sample from which the genetic material was extracted. In preferred embodiments, the subject is a human, but non-human subjects are also possible in other embodiments.

As shown in FIGS. 1-3, the methods also include extracting nucleic acids from the biological sample 110 and processing the nucleic acids for DNA sequencing, thereby generating DNA sequencing data 120. In some embodiments, DNA is extracted from a biological sample, such as a fecal sample, using the DNeasy PowerSoil Pro Kit. DNA may then be amplified using a forward primer (e.g. 5′-GTGCCAGCMGCCGCGGTAA-3′) and a reverse primer (e.g. 5′-GGACTACHVGGGTWTCTAAT-3′). A DNA library may be prepared according to the manufacturer's protocol for the illumina MiSeq. Paired-end reads of 250 bp may be sequenced using the illumina MiSeq sequencer and stored in FASTQ file format. Other methods known in the art for extracting nucleic acids from the biological sample and processing the nucleic acids for DNA sequencing to generate DNA sequencing data may also be used.

FIGS. 1-3 also show identifying, by a computer system, in the DNA sequencing data in electronic form a plurality of amplicon sequence variants (ASVs) 130. In some embodiments, the quality of sequencing data in the FASTQ file is examined using the q2-fastqc plugin. Adapters and primers may be removed with q2-cutadapt. Sequencing reads may then be processed using q2-dada2 through the following steps: trimming low-quality regions of reads, filtering out reads that are too short, merging paired-end reads into single sequences, and removing chimeric sequences. Identical sequences may be clustered into sequencing features called amplicon sequence variants (ASVs). Other methods for identifying ASVs in the electronic DNA sequencing data using a computer system may also be used.

Recording relative abundance of each ASV in a corresponding row of a relative abundance table 140 is also shown in FIGS. 1-3. The number of sequences corresponding to each ASV is counted and summarized in an abundance table, where each row represents the abundance of an ASV, and each column represents a biological sample. The sum of the abundances of all the ASVs in a biological sample is calculated for each column. The table of relative abundance is derived by dividing ASV abundances in the abundance table by the sum of all ASV abundances in each biological sample, thereby generating relative abundances. In some embodiments, the relative abundances are converted to percentages. As would be obvious to one skilled in the related arts, it would be possible to substitute columns for rows in the relative abundance table in other embodiments.

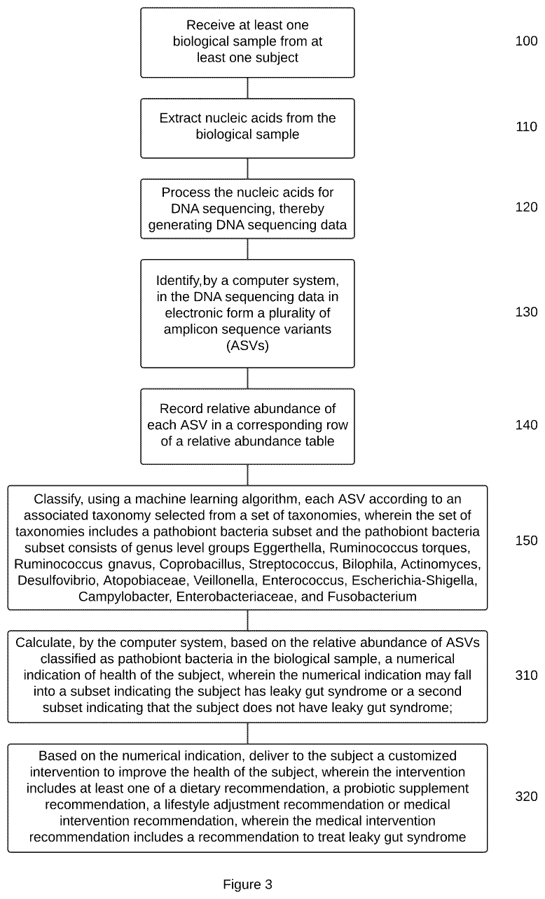

FIGS. 1 and 3 also show classifying, using a machine learning algorithm, each ASV according to an associated taxonomy selected from a set of taxonomies, wherein the set of taxonomies includes a pathobiont bacteria subset and the pathobiont bacteria subset consists of genus level groups Eggerthella, Ruminococcus torques, Ruminococcus gnavus, Coprobacillus, Streptococcus, Bilophila, Actinomyces, Desulfovibrio, Atopobiaceae, Veillonella, Enterococcus, Escherichia-Shigella, Campylobacter, Enterobacteriaceae, and Fusobacterium 150. In some embodiments, ASVs are classified based on the SILVA database, a universal database for microorganism taxonomy. For the purposes of this disclosure, the Escherichia genus is combined with the Shigella genus in a single genus level group. For the purposes of this disclosure, the Campylobacter genus level group is considered a pathobiont bacteria. In some embodiments, the set of taxonomies is the SILVA database, so ASVs may be classified based on the SILVA database, a universal database for microorganism taxonomy.

Calculating, by the computer system, based on the relative abundance of amplicon sequence variants classified as pathobiont bacteria in the biological sample, a numerical indication of health of the subject 160 is also shown in FIG. 1. In some embodiments, the numerical indication of the health of the subject may be called a gut dysbiosis score or GDS. Each subject has a microbiome profile having ASVs corresponding to a set of microbiota having relative abundances that collectively sum up to 100 percent. The microbiota can be categorized into three main types: normal microbiota, beneficial microbiota and pathobiont bacteria. Under normal circumstances, the relative abundance of normal microbiota and beneficial microbiota should be significantly higher than that of pathobiont bacteria. However, various factors such as stresses, high-fat diet, diseases, processed meat, and sugary foods, can disrupt this homeostasis, resulting in gut dysbiosis, a condition characterized by greater relative abundance of pathobiont bacteria. The relative abundances of pathobiont bacteria in the genus level groups Eggerthella, Ruminococcus torques, Ruminococcus gnavus, Coprobacillus, Streptococcus, Bilophila, Actinomyces, Desulfovibrio, Campylobacter, Atopobiaceae, Veillonella, Enterococcus, Escherichia-Shigella, Enterobacteriaceae, and Fusobacterium are used to calculate a numerical indication of the health of the subject. Higher relative abundances of these pathobiont bacteria are indicative of dysbiosis. In some embodiments, the numerical indication of the health of the subject is based on the sum, from 1 to N, of xi where each xi is the relative abundance of a bacteria (i) in the pathobiont bacteria subset consisting of genus level groups Eggerthella, Ruminococcus torques, Ruminococcus gnavus, Coprobacillus, Streptococcus, Bilophila, Actinomyces, Desulfovibrio, Atopobiaceae, Veillonella, Enterococcus, Escherichia-Shigella, Campylobacter, Enterobacteriaceae, and Fusobacterium and N is the total number of all such pathobiont bacteria contributing to dysbiosis, or

$$Numerical Indication of Health of the Subject = {\sum }_{i = 1}^{N} {x}_{i}$$

In some embodiments, the relative abundances of the pathobiont bacteria are normalized and expressed as a percentage. In some embodiments, the maximum of the sum of the relative abundances is set to a threshold below 100 percent. Some embodiments include setting a maximum threshold for a sum of the relative abundance of the amplicon sequence variants associated with the pathobiont subset. In some embodiments the methods include normalizing the relative abundances of the amplicon sequence variants associated with the pathobiont subset. One skilled in the art will be aware of multiple numerical methods, including for normalization.

In some embodiments the resulting numerical indication of health is a score ranging from 1 to 10, with a higher score indicating poorer health. In this system, a score of 1 represents a normal condition, such as, for example where the relative abundance of pathobiont bacteria is less than about 5%. Scores of 2-3, 4-7, 8-9, 10 then would indicate mild, moderate, high, and severe dysbiosis respectively, with pathobiont bacteria comprising about 5-10%, 10-20%, 20-40%, and more than about 40% of the total microbiome respectively.

In some embodiments, calculating the numerical indication includes assessing a range of the numerical indication from zero to the numerical indication based on the maximum threshold. For example, the range may be from 0 to 1, 1 to 10, 1 to 100, or any other convenient numerical range. The methods may also include associating, with a first dietary recommendation, the probiotic supplement recommendation, and the lifestyle adjustment recommendation, a first portion of the range of the numerical indication starting from zero. For example, the first dietary recommendation may be to maintain the subject's current diet, since it is associated with the first portion of the range corresponding to a normal condition. The methods may also associate, with a second dietary recommendation, the probiotic supplement recommendation, and the lifestyle adjustment recommendation a second portion of the range of the numerical indication starting from the first portion. For example, the second dietary recommendation may be to add more functional foods or reduce sugary foods in the subject's current diet, since it is associated with the second portion of the range corresponding to a mild dysbiosis. A third dietary recommendation, the probiotic supplement recommendation, and the lifestyle adjustment recommendation may be associated with a third portion of the range of the numerical indication starting from the second portion. For example, the third dietary recommendation may be to add more functional foods and reduce sugary foods in the subject's current diet, since it is associated with the third portion of the range corresponding to a moderate dysbiosis. And the methods may also associate, with a fourth dietary recommendation, the probiotic supplement recommendation, the lifestyle adjustment recommendation, and the medical intervention recommendation, a fourth portion of the range of the numerical indication starting from the third portion. For example, the fourth dietary recommendation may include foods to avoid and foods to increase, such as adding more functional foods, especially those that would help reduce harmful and pathobiont bacteria, and reduce sugary and processed foods in the subject's current diet, since it is associated with the fourth portion of the range corresponding to a high dysbiosis. A fifth dietary recommendation, the probiotic supplement recommendation, the lifestyle adjustment recommendation, and the medical intervention recommendation, may be associated with a fifth portion of the range of the numerical indication starting from the fourth portion. For example, the medical intervention recommendation may be to consult a doctor about one or more medical conditions for treatment.

FIG. 1 also shows the method step of based on the numerical indication, delivering to the subject a customized intervention to improve the health of the subject, wherein the intervention includes at least one of a dietary recommendation, a probiotic supplement recommendation, a lifestyle adjustment recommendation or medical intervention recommendation 170. The dietary recommendation may include foods to avoid and recommendations for foods to add or increase in the subject's diet. Foods to add or increase may be functional foods found in an Asian diet, including those in Table 1, below. In some embodiments, foods to avoid include some or all of the following: sugary foods, processed foods, raw foods, red meat, fast food, high fructose corn syrup, instant foods, fatty foods, sausages, bacon, grilled and roasted foods, foods containing additives, foods containing preservatives, artificial sweeteners, and foods high in saturated fats. The dietary recommendation may also include specific meals or dishes, such as dishes common in an Asian diet or a Thai diet.

TABLE 1

| Functional Foods | Function | Benefit |
|---|---|---|
| Half-Ripe Banana | Resistance Starch | Bulk up stool and pass |
| | | through your gut undigested |
| | | Serve as food for good |
| | | bacteria |
| Artichoke | Inulin \| Antioxidant | Reduce triglycerides and |
| | | total and LDL cholesterol |
| | | Lower blood pressure |
| | | Lower blood glucose |
| | | Reduce inflammation and |
| | | protect liver from damage |
| | | Boost good bacteria |
| | | Alleviate digestive |
| | | symptoms |
| | | Have anticancer effects |
| Oat (Beta-glucan) | Soluble Fiber \| Antioxidant | Reduce blood glucose and |
| | | insulin resistance |
| | | Increase good bacteria |
| Sushi Rice | Resistance Starch | Bulk up stool and pass |
| | | through your gut undigested |
| | | Serve as food for good |
| | | bacteria |
| Chilled Cooked Rice | Resistance Starch | Bulk up stool and pass |
| | | through your gut undigested |
| | | Serve as food for good |
| | | bacteria |
| Sourdough Bread | Soluble Fiber \| Polyphenols | Reduce blood glucose |
| | | Support gut health |
| | | Reduce risk of heart disease |
| | | Rich in vitamins and |
| | | minerals |
| Natto | Probiotics | Rich in protein, vitamins and |
| | | minerals |
| | | Reduce unpleasant |
| | | digestive symptoms |
| | | Help body absorb nutrients |
| | | more easily |
| | | Rich in vitamin K2 and |
| | | calcium contributing to |
| | | stronger and healthier bones |
| | | Reduce cholesterol and |
| | | blood pressure levels with |
| | | the combination of fiber, |
| | | probiotics, vitamin K2, and |
| | | nattokinase |
| | | Improve immune system |
| | | using probiotics, vitamin C |
| | | and several minerals |
| Plain Yogurt | Probiotics | source of protein, calcium, |
| | | vitamins |
| | | increase good bacteria and |
| | | reduce harmful bacteria |
| | | boost immune system |
| | | enhance absorption of |
| | | vitamins and minerals |
| Garlic (allicin) | Soluble Fiber \| Antioxidant | Reduce blood pressure |
| | | Lower risk of heart disease |
| | | Prevent Alzheimer's disease |
| | | and dementia |
| | | Detoxify heavy metals in the |
| | | body |
| | | Improve bone health |
| | | Protect against illness (cold) |
| Onion | Soluble Fiber | Reduce swelling and lung |
| | | tightness related to asthma |
| | | Reduce cholesterol |
| | | Lower blood sugar |
| Kimchi | Probiotics | source of protein, calcium, |
| | | vitamins |
| | | increase good bacteria and |
| | | reduce harmful bacteria |
| | | boost immune system |
| | | enhance absorption of |
| | | vitamins and minerals |
| Pickle | Probiotics | increase good bacteria and |
| | | reduce harmful bacteria |
| | | boost immune system |
| | | enhance absorption of |
| | | vitamins and minerals |
| Kombucha | Probiotics | increase good bacteria and |
| | | reduce harmful bacteria |
| | | boost immune system |
| | | enhance absorption of |
| | | vitamins and minerals |
| Asparagus | Insoluble Fiber \| Soluble | Bulk up stool and pass |
| | Fiber \| Flavonoid | through your gut undigested |
| | | Serve as food for good |
| | | bacteria |
| | | Anti-inflammation |
| Mushroom | Soluble Fiber | Source of vitamin D, vitamin |
| | | B6, Selenium |
| | | Selenium can help prevent |
| | | cell damage in our bodies |
| | | Vitamin D helps with cell |
| | | growth |
| | | Vitamin B6 helps our bodies |
| | | form red blood cells |
| | | Maintain a healthy immune |
| | | system |
| Tempeh | Probiotics \| Soluble Fiber | Antidiabetic |
| | | Improve cognitive functions |
| | | Lower blood cholesterol |
| | | Anti-aging |
| | | Increase gut balance |
| Curcumin in Turmeric | Antioxidant | Combat free radicals |
| Bone Broth Soup | Soluble Fiber \| Amino Acid \| | Repair and strengthen gut |
| | Collagen \| Vitamin | barrier |
| | | Source of nutrients |

The probiotic supplement recommendation may include a recommendation for a selected probiotic supplement formulated, for example, to promote general gut health, boost immunity, promote better sleep, and/or reduce visceral and liver fat. In some embodiments, the lifestyle adjustment recommendation may include a recommendation to maintain or change exercise habits, stress level, and/or sleep habits. And the medical intervention recommendation may be to consult a doctor about one or more medical conditions for treatment.

In some embodiments, the customized intervention includes a written report describing the intervention and including information about the subject, the numerical indication, information about gut microbiome homeostasis, information about gut microbiome imbalance, and information about gut microbiome diversity. The information about the subject may include only an identifier of the subject or also include demographic information and information from a questionnaire submitted with the biological sample. The numerical indication of the health of the subject may be included in the written report along with an indication of the range and the portion of the range to which the subject's numerical indication belongs. In some embodiments, the information about gut microbiome homeostasis includes information about food digestion, vitamin biosynthesis, short-chain fatty acid production, and neurotransmitter production. The information about gut microbiome imbalance may include assessment of food intolerances, gastrointestinal health, metabolic health, and degenerative disease. In some embodiments, the information about gut microbiome diversity includes the number of microbial species identified in the biological sample.

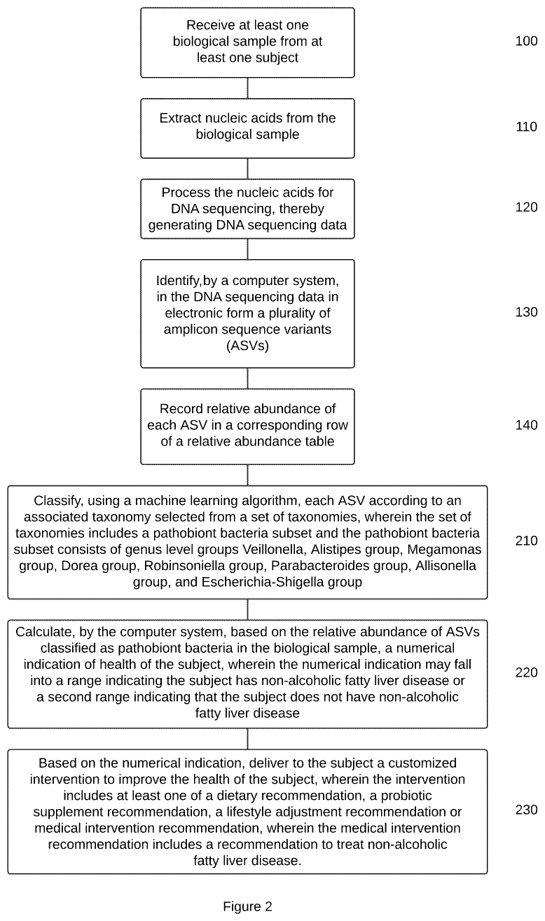

FIG. 2 shows a method of diagnosing non-alcoholic fatty liver disease. In addition to steps 100, 110, 120, 130, and 140, as described above, FIG. 2 shows classifying, using a machine learning algorithm, each amplicon sequence variant (ASV) according to an associated taxonomy selected from a set of taxonomies, wherein the set of taxonomies includes a pathobiont bacteria subset and the pathobiont bacteria subset consists of genus level groups Veillonella group, Alistipes group, Megamonas group, Dorea group, Robinsoniella group, Parabacteroides group, Allisonella group, and Escherichia-Shigella group 210. In some embodiments, ASVs are classified based on the SILVA database, a universal database for microorganism taxonomy. For the purposes of this disclosure, the Escherichia genus is combined with the Shigella genus in a single genus level group. In some embodiments, the set of taxonomies is the SILVA database, so ASVs may be classified based on the SILVA database, a universal database for microorganism taxonomy.

Calculating, by the computer system, based on the relative abundance of amplicon sequence variants classified as pathobiont bacteria in the biological sample, a numerical indication of health of the subject, wherein the numerical indication may fall into a range indicating the subject has non-alcoholic fatty liver disease or a second range indicating that the subject does not have non-alcoholic fatty liver disease 220 is also shown in FIG. 2. Each subject has a microbiome profile having ASVs corresponding to a set of microbiota having relative abundances that collectively sum up to 100 percent. The microbiota can be categorized into three main types: normal microbiota, beneficial microbiota and pathobiont bacteria. Under normal circumstances, the relative abundance of normal microbiota and beneficial microbiota should be significantly higher than that of pathobiont bacteria. However, various factors can disrupt this homeostasis, resulting in non-alcoholic fatty liver disease, a condition characterized by greater relative abundance of certain pathobiont bacteria. The relative abundances of pathobiont bacteria in the genus level groups Veillonella group, Alistipes group, Megamonas group, Dorea group, Robinsoniella group, Parabacteroides group, Allisonella group, and Escherichia-Shigella group are used to calculate the numerical indication of health of the subject. In some embodiments, the relative abundances of the selected pathobiont bacteria are normalized before summation. The normalization method may use generalized linear model. The numerical indication may fall into a range indicating the subject has non-alcoholic fatty liver disease or a second range indicating that the subject does not have non-alcoholic fatty liver disease. A mathematical model may be created using a generalized linear model (GLM), where the relative abundances of a bacterium, for example, the relative abundances of selected pathobiont bacteria, are used as the independent variable to predict a score ranging from 0 to 1. The resulting model will predict a score based on the relative abundance of the bacterium. This score is then multiplied by 100 to convert it into a percentage. One skilled in the art will be aware of alternate numerical methods, including for normalization.

In some embodiments, calculating the numerical indication includes assessing, based on a reference population, a normal range of the relative abundance of amplicon sequence variants classified as pathobiont bacteria. If the numerical indication is below the normal range then then the method provides a first dietary recommendation, the probiotic supplement recommendation, and the lifestyle adjustment recommendation. For example, the first dietary recommendation may be to add more functional foods or reduce sugary foods in the subject's current diet, since the numerical indication is below the normal range. If the numerical indication is in the normal range then the method provides a second dietary recommendation, the probiotic supplement recommendation, and the lifestyle adjustment recommendation. In cases where the numerical indication is in the normal range, for example, the second dietary recommendation and the lifestyle adjustment recommendation may be to maintain the status quo. If the numerical indication is above the normal range then the method provides a third dietary recommendation, the probiotic supplement recommendation, the lifestyle adjustment recommendation, and the medical intervention recommendation. For example, the third dietary recommendation may include foods to avoid and foods to increase, such as adding more functional foods, especially those that would help reduce harmful and pathobiont bacteria, and reduce sugary and processed foods in the subject's current diet, since the numerical indication is above the normal range.

FIG. 2 also shows based on the numerical indication, delivering to the subject a customized intervention to improve the health of the subject, wherein the intervention includes at least one of a dietary recommendation, a probiotic supplement recommendation, a lifestyle adjustment recommendation or medical intervention recommendation, wherein the medical intervention recommendation includes a recommendation to treat non-alcoholic fatty liver disease 230. The dietary recommendation may include foods to avoid and recommendations for foods to add or increase in the subject's diet. In some embodiments, the dietary recommendation is based on a Thai diet. In some embodiments, the dietary recommendation includes functional foods found in an Asian diet, such as those in Table 1, above. In some embodiments, foods to avoid include some or all of the following: sugary foods, processed foods, raw foods, red meat, fast food, high fructose corn syrup, instant foods, fatty foods, sausages, bacon, grilled and roasted foods, foods containing additives, foods containing preservatives, artificial sweeteners, and foods high in saturated fats. The dietary recommendation may also include specific meals or dishes, such as dishes common in an Asian diet or a Thai diet. The probiotic supplement recommendation may include a recommendation for a selected probiotic supplement formulated to reduce visceral and liver fat. In some embodiments, the lifestyle adjustment recommendation may include a recommendation to maintain or change exercise habits, stress level, and/or sleep habits.

In some embodiments, the customized intervention includes a written report describing the intervention and including information about the subject, the numerical indication, information about gut microbiome homeostasis, information about gut microbiome imbalance, information about gut microbiome diversity, and information about non-alcoholic fatty liver disease. The information about the subject may include only an identifier of the subject or also include demographic information and information from a questionnaire submitted with the biological sample. The numerical indication of the health of the subject may be included in the written report along with an indication of the range and the portion of the range to which the subject's numerical indication belongs. In some embodiments, the information about gut microbiome homeostasis includes information about food digestion, vitamin biosynthesis, short-chain fatty acid production, and neurotransmitter production. The information about gut microbiome imbalance may include assessment of food intolerances, gastrointestinal health, metabolic health, and degenerative disease. In some embodiments, the information about gut microbiome diversity includes the number of microbial species identified in the biological sample.

FIG. 3 shows a method of diagnosing leaky gut syndrome. In addition to steps 100, 110, 120, 130, 140, and 150 as described above, FIG. 3 shows calculating, by the computer system, based on the relative abundance of amplicon sequence variants classified as pathobiont bacteria in the biological sample, a numerical indication of health of the subject, wherein the numerical indication may fall into a subset indicating the subject has leaky gut syndrome or a second subset indicating that the subject does not have leaky gut syndrome 310. Each subject has a microbiome profile having ASVs corresponding to a set of microbiota having relative abundances that collectively sum up to 100 percent. The microbiota can be categorized into three main types: normal microbiota, beneficial microbiota and pathobiont bacteria. Under normal circumstances, the relative abundance of normal microbiota and beneficial microbiota should be significantly higher than that of pathobiont bacteria. However, various factors such as stresses, high-fat diet, diseases, processed meat, and sugary foods, can disrupt this homeostasis, resulting in leaky gut syndrome. The relative abundances of pathobiont bacteria in the genus level groups Eggerthella, Ruminococcus torques, Ruminococcus gnavus, Coprobacillus, Streptococcus, Bilophila, Actinomyces, Desulfovibrio, Campylobacter, Atopobiaceae, Veillonella, Enterococcus, Escherichia-Shigella, Enterobacteriaceae, and Fusobacterium are used to calculate a numerical indication of the health of the subject. Higher relative abundances of these pathobiont bacteria are indicative of leaky gut syndrome. In some embodiments, the method also includes evaluating the relative abundances of Bacteroides thetaiotaomicron, Enterobacteriaceae, Desulfovibrio, Bilophila, Ruminococcus gnavus, Clostridium sensu stricto 1, Lachnoclostridium, Eggerthella, Anaerotruncus, and Peptococcaceae. In some embodiments, the maximum of the sum of the relative abundances is set to a threshold below 100 percent. Some embodiments include setting a maximum threshold for a sum of the relative abundance of the amplicon sequence variants associated with the pathobiont subset. In some embodiments the methods include normalizing the relative abundances of the amplicon sequence variants associated with the pathobiont subset. One skilled in the art will be aware of alternate numerical methods, including for normalization.

In some embodiments the resulting numerical indication of health is a score ranging from 1 to 10, with a higher score indicating poorer health. In this system, a score of 1 indicates that the subject does not have leaky gut syndrome, such as, for example where the relative abundance of pathobiont bacteria is less than about 5%. Scores of 2-3, 4-7, 8-9, 10 then would indicate mild, moderate, high, and severe leaky gut syndrome respectively, with pathobiont bacteria comprising about 5-10%, 10-20%, 20-40%, and more than about 40% of the total microbiome respectively.

In some embodiments, calculating the numerical indication includes assessing a range of the numerical indication from zero to the numerical indication based on the maximum threshold. For example, the range may be from 0 to 1, 1 to 10, 1 to 100, or any other convenient numerical range. The methods may also include associating, with a first dietary recommendation, the probiotic supplement recommendation, and the lifestyle adjustment recommendation, a first portion of the range of the numerical indication starting from zero. For example, the first dietary recommendation may be to maintain the subject's current diet, since it is associated with the first portion of the range corresponding to a normal condition. The methods may also associate, with a second dietary recommendation, the probiotic supplement recommendation, and the lifestyle adjustment recommendation a second portion of the range of the numerical indication starting from the first portion. For example, the second dietary recommendation may be to add more functional foods or reduce sugary foods in the subject's current diet, since it is associated with the second portion of the range corresponding to a mild impairment. A third dietary recommendation, the probiotic supplement recommendation, and the lifestyle adjustment recommendation may be associated with a third portion of the range of the numerical indication starting from the second portion. For example, the third dietary recommendation may be to add more functional foods and reduce sugary foods in the subject's current diet, since it is associated with the third portion of the range corresponding to a moderate impairment. And the methods may also associate, with a fourth dietary recommendation, the probiotic supplement recommendation, the lifestyle adjustment recommendation, and the medical intervention recommendation, a fourth portion of the range of the numerical indication starting from the third portion. For example, the fourth dietary recommendation may include foods to avoid and foods to increase, such as adding more functional foods, especially those that would help reduce harmful and pathobiont bacteria, and reduce sugary and processed foods in the subject's current diet, since it is associated with the fourth portion of the range corresponding to a high impairment. A fifth dietary recommendation, the probiotic supplement recommendation, the lifestyle adjustment recommendation, and the medical intervention recommendation, may be associated with a fifth portion of the range of the numerical indication starting from the fourth portion. For example, the medical intervention recommendation may be to consult a doctor about leaky gut syndrome for treatment.

FIG. 3 also shows based on the numerical indication, delivering to the subject a customized intervention to improve the health of the subject, wherein the intervention includes at least one of a dietary recommendation, a probiotic supplement recommendation, a lifestyle adjustment recommendation or medical intervention recommendation, wherein the medical intervention recommendation includes a recommendation to treat leaky gut syndrome 320. The dietary recommendation may include foods to avoid and recommendations for foods to add or increase in the subject's diet. In some embodiments, the dietary recommendation is based on a Thai diet. In some embodiments, the dietary recommendation includes functional foods found in an Asian diet, such as those in Table 1, above. In some embodiments, foods to avoid include some or all of the following: sugary foods, processed foods, raw foods, red meat, fast food, high fructose corn syrup, instant foods, fatty foods, sausages, bacon, grilled and roasted foods, foods containing additives, foods containing preservatives, artificial sweeteners, and foods high in saturated fats. The dietary recommendation may also include specific meals or dishes, such as dishes common in an Asian diet or a Thai diet. The probiotic supplement recommendation may include a recommendation for a selected probiotic supplement formulated to reduce visceral and liver fat. In some embodiments, the lifestyle adjustment recommendation may include a recommendation to maintain or change exercise habits, stress level, and/or sleep habits.

In some embodiments, the customized intervention includes a written report describing the intervention and including information about the subject, the numerical indication, information about gut microbiome homeostasis, information about gut microbiome imbalance, information about gut microbiome diversity, and information about leaky gut syndrome. The information about the subject may include only an identifier of the subject or also include demographic information and information from a questionnaire submitted with the biological sample. The numerical indication of the health of the subject may be included in the written report along with an indication of the range and the portion of the range to which the subject's numerical indication belongs. In some embodiments, the information about gut microbiome homeostasis includes information about food digestion, vitamin biosynthesis, short-chain fatty acid production, and neurotransmitter production. The information about gut microbiome imbalance may include assessment of food intolerances, gastrointestinal health, metabolic health, and degenerative disease. In some embodiments, the information about gut microbiome diversity includes the number of microbial species identified in the biological sample. One skilled in the art may include other information the written report.

### Examples

The numerical indicator of the health of the subject according to the embodiment described in reference to FIG. 1, also called a gut dysbiosis score or GDS, has been tested with gut microbiota profiles of Thai individuals and then validated by comparing to age, obesity, and disease-related variables. The test population had the demographic characteristics shown in Table 2, below.

TABLE 2

| Gender | |
|---|---|
| Female | 737 |
| Male | 638 |
| Age Group | |
| <18 | 36 |
| 18-25 | 212 |
| 25-35 | 222 |
| 35-45 | 310 |
| 45-55 | 269 |
| >=55 | 312 |
| Not specified | 15 |
| BMI Group | |
| Under weight | 220 |
| Normal | 516 |
| Overweight | 152 |
| Obese | 342 |
| Very obese | 128 |
| Not specified | 19 |
| Nationality | |
| Thai | 1352 |
| Other | 24 |
| Have underlying disease? | |
| TRUE | 575 |
| FALSE | 801 |

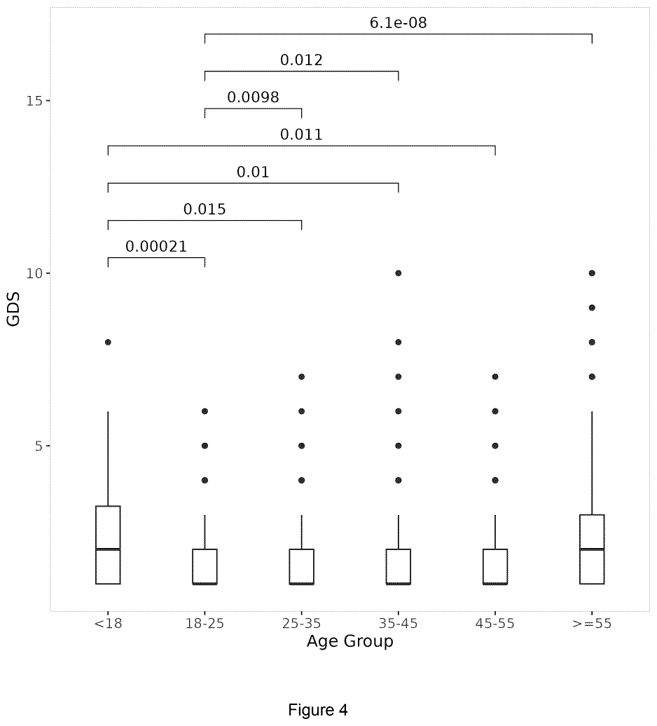

Gut dysbiosis is associated with aging. For example, Escherichia coli, which is an important marker of gut dysbiosis, is likely to increase in the elderly. Therefore, to validate the performance of the numerical indication of the health of the subject according to the embodiments described with reference to FIG. 1, stool samples from a cohort of elderly people were collected and gut microbiome profiles were made, and then the numerical indication was calculated for each elderly subject, as summarized below in Table 3 and shown in FIG. 4.

TABLE 3
Gut Dysbiosis Score (GDS) across Age Group

| Age | GDS | GDS | GDS | GDS |
|---|---|---|---|---|
| group | mean | median | minimum | maximum |
| <18 | 2.96 | 2 | 1 | 9 |
| 18-25 | 1.68 | 1 | 1 | 6 |
| 25-35 | 1.92 | 2 | 1 | 7 |
| 35-45 | 1.98 | 2 | 1 | 10 |
| 45-55 | 1.97 | 2 | 1 | 7 |
| >=55 | 2.64 | 2 | 1 | 10 |
| Not specified | 2.31 | 2 | 1 | 5 |

As shown in FIG. 4, the GDSs of elderly people aged over 55 years were found to be statistically significantly higher than those of people aged 18-25 years (p-value=5.2×10⁻¹⁰) and tended to be higher than those of other adult age groups, including the age groups for 25-35, 35-45, and 45-55 year-olds. The GDSs of young people aged 18-25 years were found to be statistically significantly greater than those of middle-aged people aged 25-35 (p-value=0.01) and 35-45 years (p-value=0.00037). However, GDSs of children and adolescents under 18 were statistically significantly worse than older people aged 18-25 (p-value=2.4×10⁻⁵), 25-35 (p-value=0.0024), 35-45 (p-value=0.0041), and 45-55 years (p-value=0.0048). This is not surprising because children and adolescents often consume processed meats such as sausages, ham, Chinese sausages, fish balls, and other processed foods that can lead to gut dysbiosis.

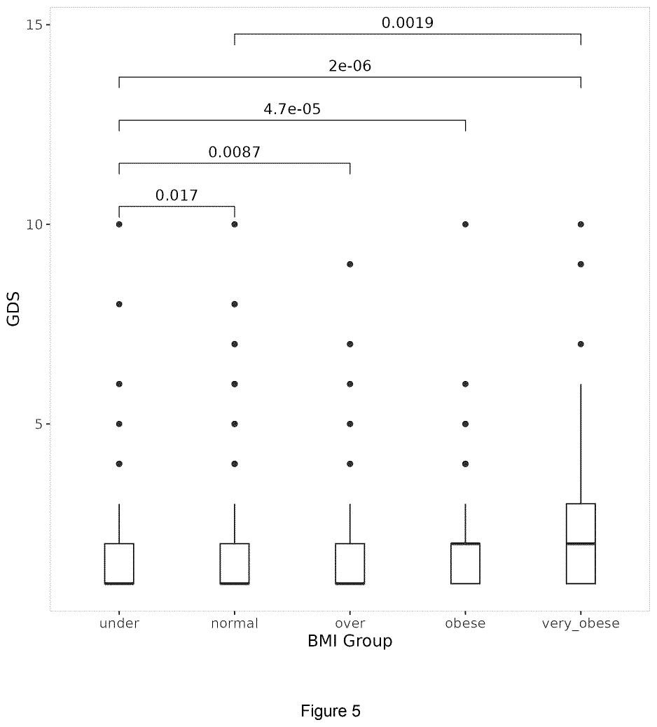

As shown in FIG. 5, the numerical indications of the health of the subject according to the embodiments described with reference to FIG. 1 (GDSs) for underweight people were statistically significantly lower than those of normal-weight (p-value=0.0074), overweight (p-value=0.003), obese (p-value=1.5×10⁻⁵), and very obese people (p-value=3.8×10⁻⁶). The GDSs of normal-weight people were statistically significantly smaller than those of obese people (p-value=0.0049). Thus both underweight and normal weight people tend to have lower gut dysbiosis scores (numerical indicators) as calculated by the embodiments described with reference to FIG. 1 than higher weight people.

The correlation between GDS and different age groups suggests that GDS can serve as an indicator of biological aging. GDS is useful to assess the extent of gut dysbiosis, guiding personalized treatments such as dietary modifications, probiotic or prebiotic supplementation, or specific medications. In some embodiments, for mild gut dysbiosis, dietary adjustments are typically recommended. In some embodiments, for cases of moderate dysbiosis, both dietary changes and probiotic or prebiotic supplements may be advised. In some embodiments, for high or severe gut dysbiosis level, medical consultation is necessary to determine appropriate interventions. Furthermore, the numerical indicator disclosed herein can indicate the risk for gastrointestinal problems such as leaky gut syndrome, gut inflammation, excessive gas production. It also highlights the potential for a subject to develop metabolic conditions, including non-alcoholic fatty liver disease, obesity, type 2 diabetes, and the like.

It is understood that the preceding description is given merely by way of illustration and not in limitation of the invention and that various modifications may be made thereto without departing from the spirit of the invention as claimed.

In understanding the scope of the present invention, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “having” and “including” and their derivatives. Also, the terms “section, “part,” “portion,” “member” or “element” when used in the singular can have the dual meaning of a single part or a plurality of parts.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

1. A method comprising:
receiving at least one biological sample from at least one subject;
extracting nucleic acids from the biological sample;
processing the nucleic acids for DNA sequencing, thereby generating DNA sequencing data;
identifying, by a computer system, in the DNA sequencing data in electronic form a plurality of amplicon sequence variants;
recording relative abundance of each amplicon sequence variant in a corresponding row of a relative abundance table;
classifying, using a machine learning algorithm, each amplicon sequence variant according to an associated taxonomy selected from a set of taxonomies, wherein the set of taxonomies includes a pathobiont bacteria subset and the pathobiont bacteria subset consists of genus level groups Eggerthella group, Ruminococcus torques group, Ruminococcus gnavus group, Coprobacillus group, Streptococcus group, Bilophila group, Actinomyces group, Desulfovibrio group, Atopobiaceae group, Veillonella group, Enterococcus group, Escherichia-Shigella group, Campylobacter group, Enterobacteriaceae, and Fusobacterium;
calculating, by the computer system, based on the relative abundance of amplicon sequence variants classified as pathobiont bacteria in the biological sample, a numerical indication of health of the subject;
based on the numerical indication, delivering to the subject a customized intervention to improve the health of the subject, wherein the intervention includes at least one of a dietary recommendation, a probiotic supplement recommendation, a lifestyle adjustment recommendation or medical intervention recommendation.

2. The method of claim 1 wherein the subject is a human.

3. The method of claim 1 wherein the biological sample is a stool sample.

4. The method of claim 1, wherein the customized intervention includes a written report describing the intervention and including information about the subject, the numerical indication, information about gut microbiome homeostasis, information about gut microbiome imbalance, and information about gut microbiome diversity.

5. The method of claim 1, including setting a maximum threshold for a sum of the relative abundance of the amplicon sequence variants associated with the pathobiont subset.

6. The method of claim 5, wherein the calculating the numerical indication includes assessing a range of the numerical indication from zero to the numerical indication based on the maximum threshold; and further including
associating, with a first dietary recommendation, the probiotic supplement recommendation, and the lifestyle adjustment recommendation, a first portion of the range of the numerical indication starting from zero;
associating, with a second dietary recommendation, the probiotic supplement recommendation, and the lifestyle adjustment recommendation a second portion of the range of the numerical indication starting from the first portion;
associating, with a third dietary recommendation, the probiotic supplement recommendation, and the lifestyle adjustment recommendation, a third portion of the range of the numerical indication starting from the second portion;
associating, with a fourth dietary recommendation, the probiotic supplement recommendation, the lifestyle adjustment recommendation, and the medical intervention recommendation, a fourth portion of the range of the numerical indication starting from the third portion; and
associating, with a fifth dietary recommendation, the probiotic supplement recommendation, the lifestyle adjustment recommendation, and the medical intervention recommendation, a fifth portion of the range of the numerical indication starting from the fourth portion.

7. The method of claim 1, including normalizing the relative abundances of the amplicon sequence variants associated with the pathobiont subset.

8. A method of diagnosing non-alcoholic fatty liver disease comprising:
receiving at least one biological sample from at least one subject;
extracting nucleic acids from the biological sample;
processing the nucleic acids for DNA sequencing, thereby generating DNA sequencing data;
identifying, by a computer system, in the DNA sequencing data in electronic form a plurality of amplicon sequence variants;
recording relative abundance of each amplicon sequence variant in a corresponding row of a relative abundance table;
classifying, using a machine learning algorithm, each amplicon sequence variant according to an associated taxonomy selected from a set of taxonomies, wherein the set of taxonomies includes a pathobiont bacteria subset and the pathobiont bacteria subset consists of genus level groups Veillonella group, Alistipes group, Megamonas group, Dorea group, Robinsoniella group, Parabacteroides group, Allisonella group, and Escherichia-Shigella group;
calculating, by the computer system, based on the relative abundance of amplicon sequence variants classified as pathobiont bacteria in the biological sample, a numerical indication of health of the subject, wherein the numerical indication may fall into a range indicating the subject has non-alcoholic fatty liver disease or a second range indicating that the subject does not have non-alcoholic fatty liver disease;
based on the numerical indication, delivering to the subject a customized intervention to improve the health of the subject, wherein the intervention includes at least one of a dietary recommendation, a probiotic supplement recommendation, a lifestyle adjustment recommendation or medical intervention recommendation, wherein the medical intervention recommendation includes a recommendation to treat non-alcoholic fatty liver disease.

9. The method of claim 8 wherein the subject is a human and the biological sample is a stool sample.

10. The method of claim 8 wherein the dietary recommendation is based on a Thai diet.

11. The method of claim 8 wherein the dietary recommendation includes functional foods found in an Asian diet.

12. The method of claim 8, wherein the customized intervention includes a written report describing the intervention and including information about the subject, the numerical indication, information about gut microbiome homeostasis, information about gut microbiome imbalance, information about gut microbiome diversity, and information about non-alcoholic fatty liver disease.

13. The method of claim 8, wherein the calculating the numerical indication includes assessing, based on a reference population, a normal range of the relative abundance of amplicon sequence variants classified as pathobiont bacteria; and further including if the numerical indication is below the normal range then providing a first dietary recommendation, the probiotic supplement recommendation, and the lifestyle adjustment recommendation;
if the numerical indication is in the normal range then providing a second dietary recommendation, the probiotic supplement recommendation, and the lifestyle adjustment recommendation; and
if the numerical indication is above the normal range then providing a third dietary recommendation, the probiotic supplement recommendation, the lifestyle adjustment recommendation, and the medical intervention recommendation.

14. A method of diagnosing leaky gut syndrome comprising:
receiving at least one biological sample from at least one subject;
extracting nucleic acids from the biological sample;
processing the nucleic acids for DNA sequencing, thereby generating DNA sequencing data;
identifying, by a computer system, in the DNA sequencing data in electronic form a plurality of amplicon sequence variants;
recording relative abundance of each amplicon sequence variant in a corresponding row of a relative abundance table;
classifying, using a machine learning algorithm, each amplicon sequence variant according to an associated taxonomy selected from a set of taxonomies, wherein the set of taxonomies includes a pathobiont bacteria subset and the pathobiont bacteria subset consists of genus level groups Eggerthella group, Ruminococcus torques group, Ruminococcus gnavus group, Coprobacillus group, Streptococcus group, Bilophila group, Actinomyces group, Desulfovibrio group, Atopobiaceae group, Veillonella group, Enterococcus group, Escherichia-Shigella group, Campylobacter group, Enterobacteriaceae group, and Fusobacterium group;
calculating, by the computer system, based on the relative abundance of amplicon sequence variants classified as pathobiont bacteria in the biological sample, a numerical indication of health of the subject, wherein the numerical indication may fall into a subset indicating the subject has leaky gut syndrome or a second subset indicating that the subject does not have leaky gut syndrome;
based on the numerical indication, delivering to the subject a customized intervention to improve the health of the subject, wherein the intervention includes at least one of a dietary recommendation, a probiotic supplement recommendation, a lifestyle adjustment recommendation or medical intervention recommendation, wherein the medical intervention recommendation includes a recommendation to treat leaky gut syndrome.

15. The method of claim 14 wherein the subject is a human and the biological sample is a stool sample.

16. The method of claim 14, wherein the customized intervention includes a written report describing the intervention and including information about the subject, the numerical indication, information about gut microbiome homeostasis, information about gut microbiome imbalance, information about gut microbiome diversity, and information about leaky gut syndrome.

17. The method of claim 14, including setting a maximum of a sum of the relative abundance of the amplicon sequence variants associated with the pathobiont subset.

18. The method of claim 17, wherein the calculating the numerical indication includes assessing a range of the numerical indication from zero to the numerical indication based on the maximum of the sum of the relative abundance; and further including
associating, with a first dietary recommendation, the probiotic supplement recommendation, and the lifestyle adjustment recommendation, a first portion of the range of the numerical indication starting from zero;
associating, with a second dietary recommendation, the probiotic supplement recommendation, and the lifestyle adjustment recommendation, a second portion of the range of the numerical indication starting from the first portion;
associating, with a third dietary recommendation, the probiotic supplement recommendation, and the lifestyle adjustment recommendation, a third portion of the range of the numerical indication starting from the second portion;
associating, with a fourth dietary recommendation, the probiotic supplement recommendation, the lifestyle adjustment recommendation, and the medical intervention recommendation, a fourth portion of the range of the numerical indication starting from the third portion; and
associating, with a fifth dietary recommendation, the probiotic supplement recommendation, the lifestyle adjustment recommendation, and the medical intervention recommendation, a fifth portion of the range of the numerical indication starting from the fourth portion.

19. The method of claim 14, including normalizing a sum of the relative abundance of the amplicon sequence variants associated with the pathobiont subset.

20. The method of claim 14, including evaluating the relative abundances of Bacteroides thetaiotaomicron, Enterobacteriaceae, Desulfovibrio, Bilophila, Ruminococcus gnavus, Clostridium sensu stricto 1, Lachnoclostridium, Eggerthella, Anaerotruncus, and Peptococcaceae.
